(19) United States
(12) Patent Application Publication
(10) Pub. No.: US 2013/0177966 A1
(43) Pub. Date: Jul. 11, 2013

(54) METHOD AND PLANT FOR THE CULTIVATION OF PHOTOSYNTHETIC MICRO-ORGANISM

(71) Applicant: Fabrizio De Poli, Roma (IT)

(72) Inventor: Fabrizio De Poli, Roma (IT)

(73) Assignee: MAFA AMBIENTE SRL, Roma (IT)

(21) Appl. No.: 13/823,233

(22) PCT Filed: Oct. 11, 2011
(86) PCT No.: PCT/IT2011/000362

§ 371 Date: Mar. 14, 2013

(30) Foreign Application Priority Data
Nov. 4, 2010 (IT) RM2010A000584

(51) Int. Cl. C12M 1/00; C12N 1/20; C12N 1/12

(52) U.S. Cl. CPC C12M 21/02; C12N 1/12; C12N 1/20
USPC 435/252.1; 435/257.1; 435/292.1

(57) ABSTRACT

A method for cultivation of photosynthetic micro-organisms such as micro-algae, photobacteria, and similar organisms, through circulation within tubes made of transparent plastic material of the suspension of said organisms in an appropriate culture medium, envisages: carrying out agitation/mixing of said suspension in the culture medium in said tubes in a pulsed and non-continuous way; separating the biomass produced from the culture medium by means of sifting with a system of differential-inclination sieves; introducing C02 with total dissolution thereof inside the tubes in which the suspension of said micro-organisms in their own culture medium circulates, introducing also buffer solutions in said culture medium; extracting the oxygen produced in the photosynthesis in a natural way by means of skimmers distributed along the tubes traversed by the culture medium; controlling the temperature inside the tubes in order not to reach temperatures that are harmful to the growth of the photosynthetic micro-organisms by modifying the concentration of the biomass in the culture medium by means of said system of sieves so as to increase the transparency of the culture medium and reduce the solar energy absorbed.

### SUBJECT AND PURPOSES OF THE INVENTION

The subject of the present invention is a method and corresponding system for cultivation of photosynthetic micro-organisms aimed at providing energy, chemical, foodstuff, and fine-chemistry products, and at biological fixing of carbon dioxide.

According to the invention, the cultivation takes place in a closed tubular photobioreactor, designed to contain a culture medium or broth, which is equipped with systems for movement of the culture broth and for collection of the biomass, which form an integral part of the system itself.

### State of the Art in the Field of Cultivation of Photosynthetic Micro-Organisms

The cultivation of algae and other photosynthetic micro-organisms is currently a topic of particular importance, since it constitutes the preferential way for basically organic conversion of solar energy.

The efficiency of the system of photosynthesis of aquatic micro-organisms is clearly higher than the efficiency that can be achieved with plants traditionally cultivated on land, with a high production of biomass. Said high production simultaneously entails a higher and more complete fixation of CO₂, with reduction of climate-altering emissions.

There exist multiple activities of massive cultivation of photosynthetic micro-algae or bacteria, such as Spirulina maximum, Spirulina platensis, Dunaliella salina, Botrycoccus braunii, Chlorella vulgaris, Chlorella pyrenoidosa, Serenastrum capricomutum, Scenedesmus auadricauda, Porphyridium cruentum, Scenedesmus acutus, Dunaliella sp., Scenedesmus obliquus, Anabaenopsis Aulostra, Cylindrospermum, Scenecoccus sp., Scenecosystis sp., and Tolypothrix. Said activities are aimed, in the majority of cases, at the supply of products with high added value or of fine-chemistry products. However, the high costs of production and a series of problems of a technical nature have up to now limited the diffusion of systems designed to the make available animal feed or energy products.

The general techniques of production of the aforesaid micro-algae basically consist in suspending at an appropriate concentration the cells in a purposely provided liquid culture medium, in the presence of carbon dioxide and light radiation, commonly solar radiation.

The cultivation currently takes place prevalently in open baths, which present, however, numerous problems from the biological standpoint and from the standpoint of management. In fact, these open systems are sensitive to contamination of other species of algae or of harmful animals so that only algae with specific requisites for their development can be cultivated. Thus, for example, the alga Dunaliella is cultivated for the production of beta-carotene in saline conditions, which are not acceptable for the majority of other organisms.

From the economic standpoint, the cost of the production of biomass from algae is rather high (more of 2000 USD per tonne) so that a commercial production for many applications, especially in the energy sector or in the transport sector, is not practicable.

There have been numerous proposals of photobioreactors, in which the organisms are cultivated in closed tubes or bags with a high surface-to-volume ratio, which are made of various transparent materials to enable the sunlight to penetrate in the culture medium thus supplying the energy required by the micro-organisms for fixation of the carbon dioxide in the organic molecules.

The types that have been studied in greatest depth are two: horizontal photobioreactors, which consist of one or more closed horizontal tubes, and vertical reactors with mixing of air (bubble column), to which there belong, for example, the annular reactors forming the subject of the patent No. WO 2004/074423, which enable considerable reduction of the spaces used and the volumes of culture to be managed. However, said reactors are generally used for limited levels of production and have not yet been applied to cultures on a wide scale and over extensive surfaces on account of a series of problems of scaling-up, which have not yet been solved.

In particular, both in the case of cultivations in baths and in the case of photobioreactors, still to be solved are the problems regarding agitation/mixing/movement of the culture broth, when this action is carried out over extensive surfaces, and if the overall energy balance is considered.

Moreover, the problem of concentration/collection of the biomass, the density of which inside the culture is much lower than the one that is encountered in similar industrial processes, still remains to be solved.

A system of culture of photosynthetic micro-organisms in horizontal tubular photobioreactors has already been described in its general lines in the Italian patent No. 1,094,286 dated Mar. 23, 1978 (inventors L. Biondi, F. De Poli, A. Di Corato, G. Veronica—Procedimento per favorire la crescita di microoraganismi fotosintetici e organismi simili—Process for favouring growth of photosynthetic micro-organisms and similar organisms).

According to said patent, in order to prevent onset of thermal regimes markedly different from the optimal ones inside the culture tube, especially at night where the dispersion of heat through the wall of the tubes can balance the contribution of heat through daytime solar irradiation, it is envisaged to arrange externally and coaxially to the tube made of transparent plastic material that contains the culture broth a second tube made of the same or of a different transparent plastic material having a larger diameter and a smaller thickness and providing in the external tube appropriate joints, which are not necessarily fluid-tight, so as to obtain between the two tubes a gap full of stagnant air. The culture is thus contained in a sort of solar collector in which the greenhouse effect is exploited.

A culture system of the above sort has not, however, been able to solve a series of other problems, linked, for example, to the movement of the biomass in the culture broth and to the regulation of the amount of energy absorbed by the broth itself, which have in effect prevented up to now effective use thereof.

The task of the present invention is to make to a tubular reactor, whether this uses a simple tube or a coaxial tube, modifications and innovations that will be able to overcome in combination the problems that have been encountered in the production of this type of systems.

According to the present invention, a method is provided designed to favour growth of photosynthetic micro-organisms and in particular micro-algae, photobacteria, and similar organisms in a closed photobioreactor, through circulation, within tubes made of transparent plastic material, of the suspension of said organisms in an appropriate culture medium, said method being characterized in that it envisages:

carrying out agitation/mixing of the culture broth in said photobioreactor in a pulsed and non-continuous way, guaranteeing high efficiency and low levels of energy consumption;

separating the biomass produced from the culture broth by means of sifting with a system of differential-inclination sieves, guaranteeing high efficiency, low cost, and minimal levels of energy consumption;

introducing CO₂ with total dissolution thereof inside the tubular system in which the suspension circulates in its culture medium, also introducing buffering means in said culture medium;

extracting the oxygen produced in the photosynthesis at a low cost in a natural way by means of skimmers distributed along the tubular system traversed by the culture medium; and

controlling the temperature inside the tubular system of the photobioreactor in order not to reach temperatures that are harmful to the growth of micro-organisms modifying the concentration of the biomass in the culture broth by means of said system of sieves so as to increase the transparency of the culture and reduce the solar energy absorbed.

Forming an integral part of the present invention is a system constituted by a closed photobioreactor for the cultivation of photosynthetic micro-organisms, in particular micro-algae, photobacteria, and similar organisms, and by the means able to implement the method referred to above.

We shall now examine in detail said innovative characteristics and the problems jointly solved thereby.

### System for Agitation and Movement of the Biomass

All systems for cultivation of photosynthetic micro-organisms envisage a system for agitation of the biomass so as to enable the individual cells to come into contact with light in an appropriate amount. An excess of light results, in fact, in a loss of production, owing to the incapacity of the alga to absorb the maximum amount of energy, and possibly owing to photoinhibition, whereas self-shading of the culture produces areas with low photosynthetic activity, with consequent degradation of the culture. The agitation system normally also enables movement of the biomass to allow a flow that will afford proper growth and collection thereof.

In open systems (baths) this is normally obtained with systems of rotary blades. In tubular systems recourse is, instead, had to pumping systems, which must keep a high speed of flow to prevent sedimentation or flotation of the biomass. Said high speed must be transmitted along tubes having a length of even more than some kilometres, with consequent marked expenditure of energy, while the cultivated organisms must pass a number of times through pumps which can damage their structures.

According to the present invention, the movement takes place, instead, in a pulsed way, with fast displacements of the water column followed by more or less long times of stoppage of the circulation. The intensity and frequency of the pulses depends upon the apparent density of the cultivated organism (which can be higher or lower than that of the culture broth, with consequent tendency to sedimentation or to flotation, also following upon the presence, in some cases, of gaseous vesicles or of lipidic granules inside the organisms). Indicatively, we can assume one or more pulses per hour of the duration of 2-3 minutes, with a reduction of the levels of energy consumption by at least one order of magnitude.

The pulse can be generated in different ways:
through a timed pump of large dimensions, or else
by filling reservoirs set in a higher position with respect to the plane of lie of the photobioreactor, which are discharged through timed opening of valves, or by means of self-priming siphons; the latter solution is the one that normally presents lower costs and higher efficiency.

The pulse transmitted to the cultivation tube generates a turbulent movement that agitates the entire culture also at considerable distances from the point of introduction, enabling an optimal mixing of the culture and detachment of possible coatings formed by micro-organisms that have deposited along the walls.

### Collection of the Biomass

The biomass can be collected with different systems, borrowed from similar industrial processes, such as centrifugation, flotation, flocculation, or filtration. However, all these systems, which are already commercially available, are far from readily applicable to cultures the density of which is of the order of a few grams per litre.

According to a peculiar characteristic of the invention, the system proposed by the present invention envisages, instead, sifting by means of variable-inclination sieves, made of non-clogging filtering fabric.

Advantageously, the sieves (two or more, arranged in succession) have different inclinations: the first has a gentler inclination (indicatively 10-15°) in order to enable a high draining of the culture medium and a preconcentration of the biomass, which slides on towards the next frame; this has a steeper inclination (indicatively 30-80°), because the material fed thereto has already been concentrated, and the treatment of a lower flow is hence required, whilst the natural descent of the biomass, which is increasingly concentrated, requires increasingly steep slopes.

The number of the sieves and the mesh of the sieves depend upon the size of the organisms cultivated and must be adapted to the species chosen.

According to a further characteristic of the invention, these sieves can moreover perform the function of selective separation both of the younger forms from the more mature ones and of possible undesirable species that may have contaminated the culture.

### Absorption of CO₂

Since the photosynthetic production takes place through fixation of CO₂, the latter, which is normally supplied to the culture itself, must remain available for absorption by the organisms.

According to the invention, in order to facilitate absorption of CO₂ in the culture medium, recourse is had to the strategy of introducing into the culture broth a carbonate-bicarbonate buffer, aimed at increasing absorption of CO₂, such as, for example, a sodium carbonate-sodium bicarbonate buffer. In this way, since it cannot be dispersed into the atmosphere, all the CO₂ blown into the tubes as nutrient is completely absorbed by the micro-organisms.

### Removal of Oxygen from the Culture

The photosynthesis also produces large amounts of oxygen, which must be removed from the culture both for hydraulic reasons (formation of pockets of gas that slow down circulation) and to prevent a possible biological inhibition of the process, as reported by a number of authors.

In the system described, the extraction of oxygen takes place in a natural way in some stretches of the photobioreactor by resorting to simple skimmers connected to expansion vessels to prevent outflow of liquid following upon the agitation pulses.

### Control of the Temperature

One of the major problems that remain unsolved in cultures in tubular reactors is the excessively high temperature reached in the summer season and in the times of day of greater insolation since high temperatures can in many cases cause the death of the culture itself.

According to the present invention, it is envisaged to control the temperature by modifying the concentration of biomass through the choice of the separation sieves. In fact, by selecting appropriately the size of the mesh and the number of sieves, it is possible to reduce the concentration of biomass and consequently increase the transparency of the culture, with consequent reduction of the amount of energy absorbed by the culture broth.

### Plastic Materials Used

The higher cost of this type of system is constituted by the active part of the system, namely, by the plastic tubing that constitutes the collector. Advantageously, the present invention hence envisages the possibility of making the tubes of the system, as an alternative to using virgin plastic, with the use of recycled plastic, with particular reference to PET (polyethylene terephthalate), of which large amounts are available deriving from recovery of material of bottles for liquid foodstuffs (water, effervescent beverages), characterized by a high transparency and resistance to physical agents. The creation of a market for these materials falls perfectly within the objectives of the European Community of increasing the percentage of recycling of waste, while their use for non-alimentary purposes renders less stringent the aspects regarding possible contamination of the recycled material.

### DETAILED DESCRIPTION OF THE INVENTION

Further characteristics and advantages of the present invention will emerge clearly from the ensuing description on the basis of the attached plates of drawings, which illustrate purely by way of non-limiting example a preferred embodiment of the invention.

In the plates of drawings:

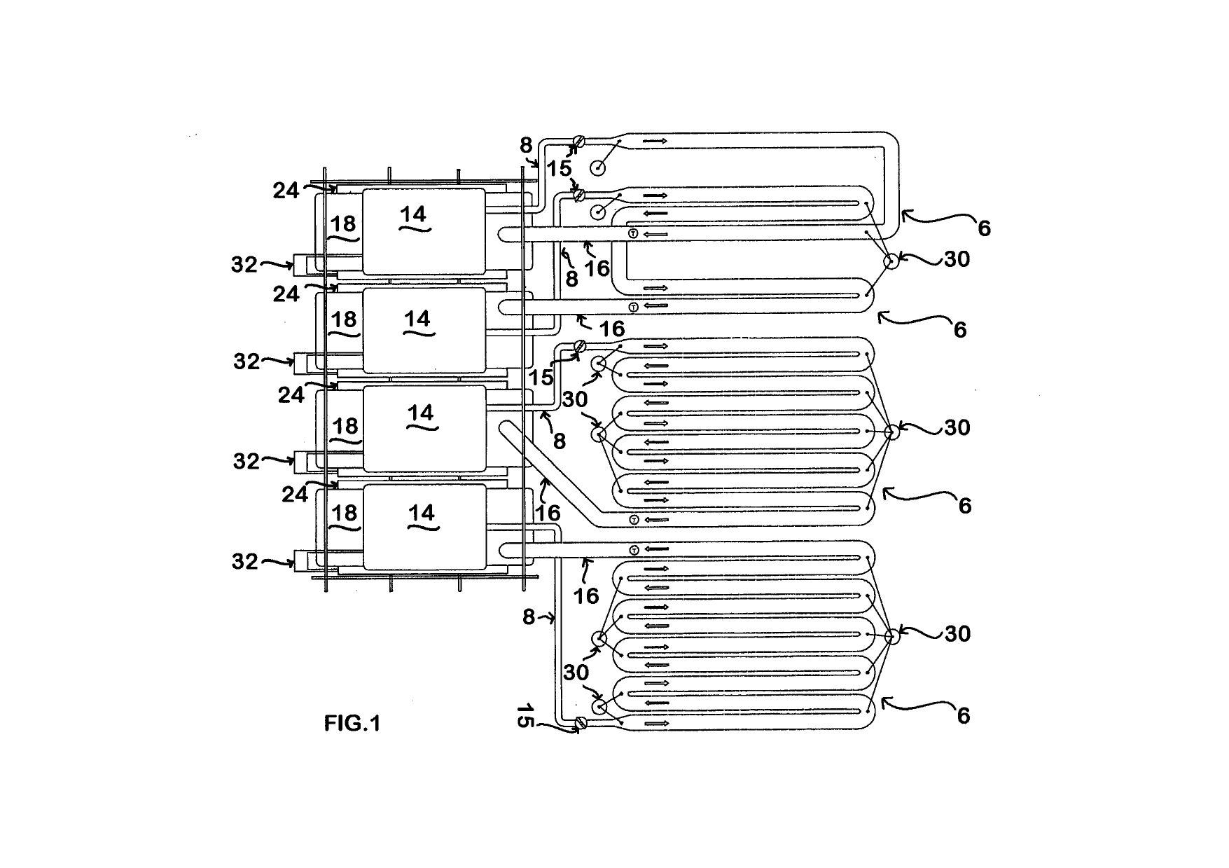

FIG. 1 is a plan view of an experimental installation used for cultivation of micro-algae comprising a plurality of circuits;

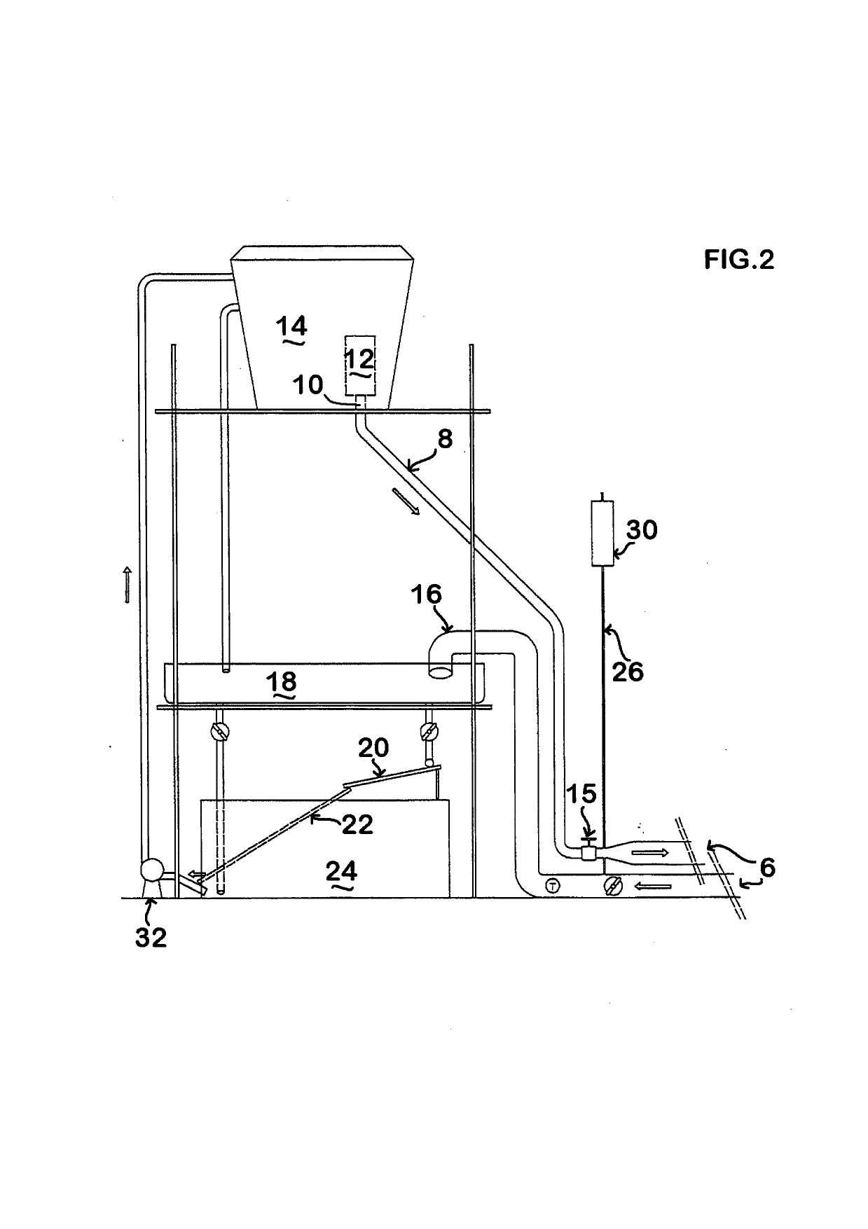

FIG. 2 is a schematic side view of the installation of FIG. 1;

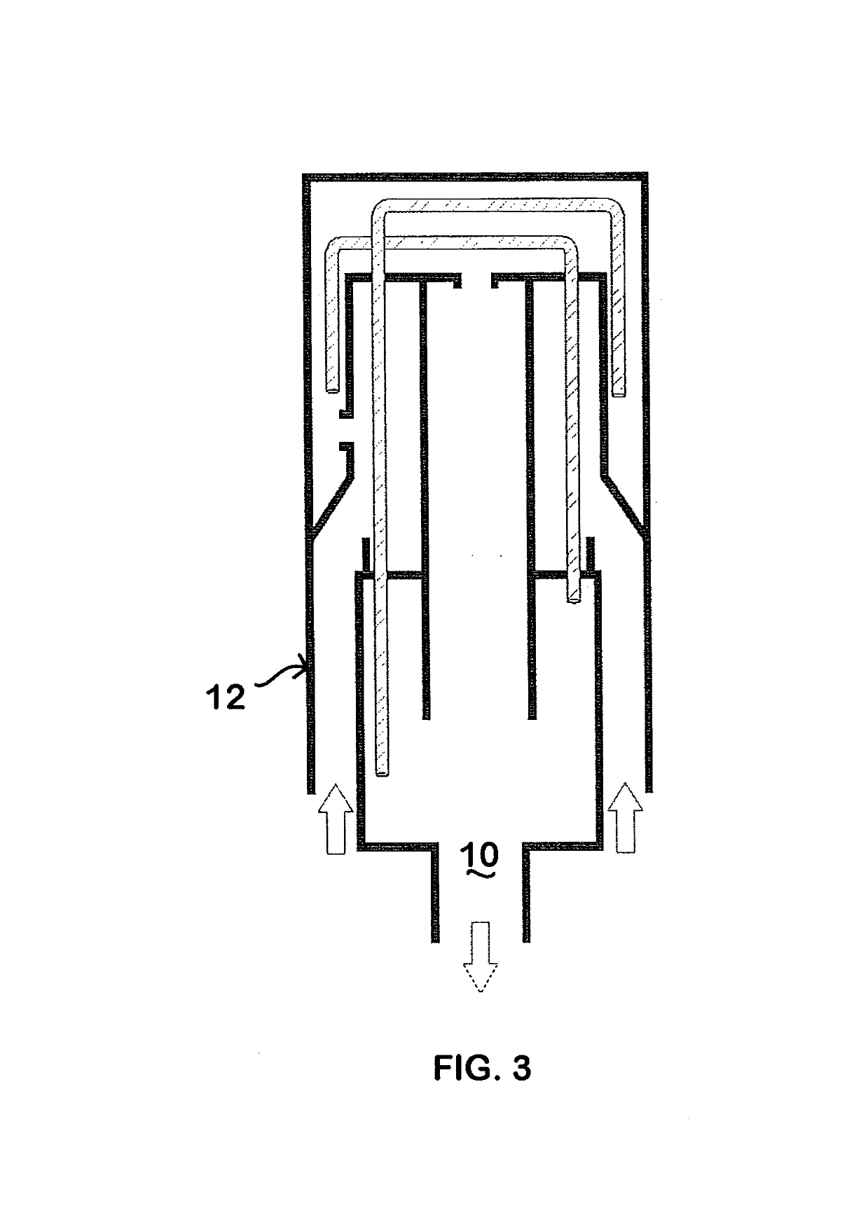

FIG. 3 is a cross-sectional view of a self-priming siphon;

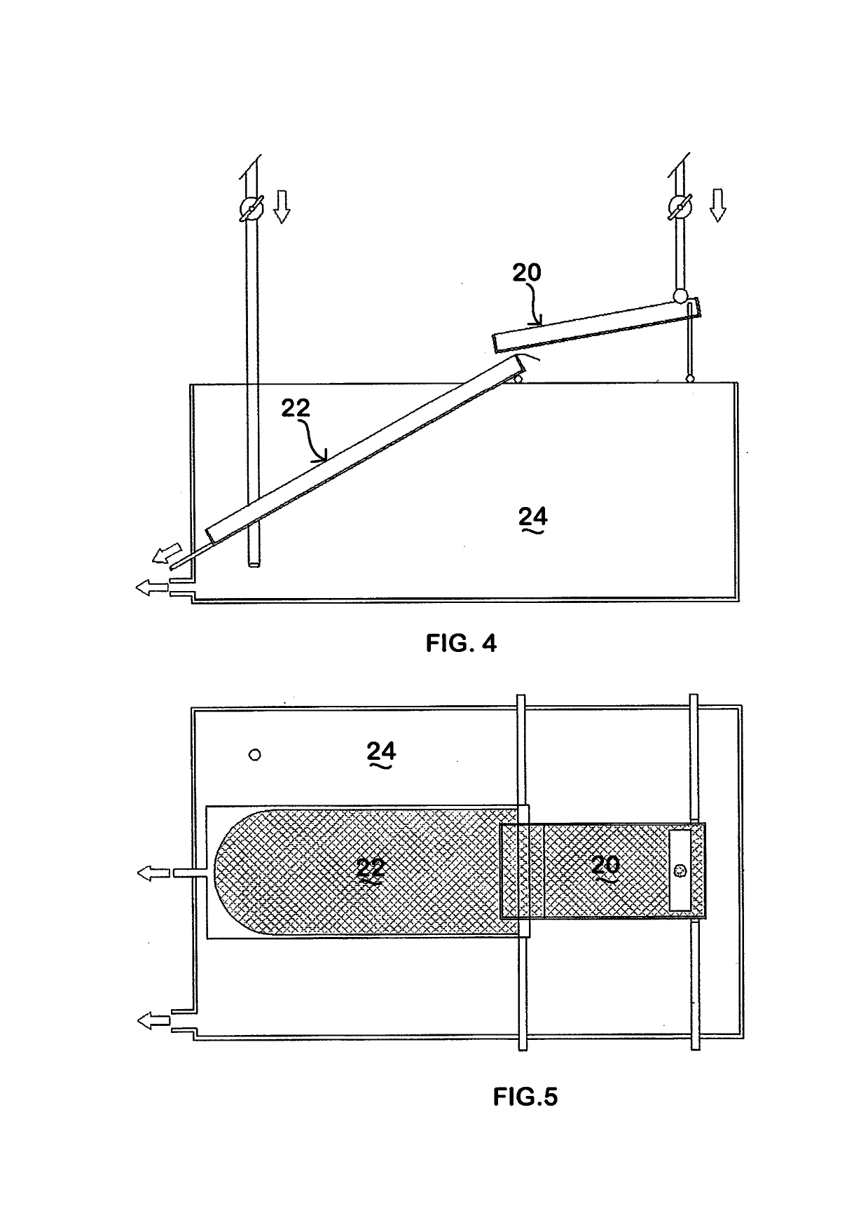

FIG. 4 is a side view of a collection bath containing the filter used for thickening the alga; and

FIG. 5 is a plan view of the bath of FIG. 4.

With reference to the figures, the apparatus for cultivation of photosynthetic micro-organisms according to the invention is constituted by a plurality of horizontal tubular coils 6, preferably made of recycled plastic, which rest on a sheet of white plastic lying on the ground. The average length of each branch of coil can range from a few tens of metres to hundreds of metres.

The delivery tube 8 of each coil unit is connected to the discharge tube 10 of a self-priming siphon 12 of a conventional type, positioned inside a charging bath 14 set at a higher position with respect to the plane of lie of the coil tube 6. Once the bath 14 has been filled with water, it is discharged by means of said self-priming siphon 12 or alternatively, by means of timed opening of a valve 14.

The outlet tube 16 of each coil unit 6 reaches, instead, a bath 18 for distribution of the culture, which is set at an intermediate level between that of the bath 14 and that of the filtering assembly. The latter is constituted by two sieves 20, 22 having different inclinations; the first sieve 20 has an inclination of about 10-15° to enable an initial preconcentration of the biomass, and the second sieve 22 has an inclination of about 30-80° to facilitate natural descent of the biomass, which is increasingly concentrated, out of the bath 24 for collection of the culture broth, positioned at the level of the plane of lie, for use thereof. The culture broth is then sent back from the bath 24 to the charging bath 14 through a pump 32.

Advantageously, the oxygen produced during photosynthesis is extracted by means of simple skimmers 26, which are connected to expansion vessels 30 to prevent the liquid from possibly coming out following upon the agitation pulses.

From what has been described so far, it is evident how the system for cultivation of photosynthetic micro-organisms according to the present invention, by resorting to a pulsed movement of the culture medium, appropriately buffered to facilitate absorption of CO₂, in combination with the use of differential-inclination sieves for selective separation of the biomass produced and for control of the temperature and of simple skimmers to remove the oxygen produced along the culture path, enable solution of all the problems that had up to now hindered effective use of a photosynthetic system with horizontal development for the culture of photosynthetic micro-organisms and constitutes an important step forwards in the sustainability of the production of bio-energy.

1. A method for cultivation of photosynthetic micro-organisms such as micro-algae, photobacteria, and similar organisms, through the circulation within tubes made of transparent plastic material of the suspension of said organisms in an appropriate culture medium, said method comprising:
carrying out agitation/mixing of said suspension in the culture medium in said tubes in a pulsed and non-continuous way;
separating the biomass produced from the culture medium by means of sifting with a system of differential-inclination sieves;
introducing CO₂ with total dissolution thereof, inside the tubes in which the suspension of said micro-organisms in their own culture medium circulates, introducing also buffer solutions in said culture medium;
extracting the oxygen produced in the photosynthesis in a natural way by means of skimmers distributed along the tubes traversed by the culture medium; and
controlling the temperature inside the tubes in order not to reach temperatures that are harmful to the growth of the photosynthetic micro-organisms by modifying the concentration of the biomass in the culture medium by means of said system of sieves so as to increase the transparency of the culture medium and reduce the solar energy absorbed.

2. The method of claim 1, wherein agitation and mixing of the culture medium in the transparent tubes take place by means of a pulsed system, obtained through timed-pumping systems or through intermittent emptying of containers set at a higher level than the plane of lie of the tubes themselves.

3. The method of claim 2, wherein the pulsed flow takes place by using a self-priming siphon, which enables fast emptying of containers set at a higher level than the tubes traversed by the culture medium.

4. The method of claim 2, wherein the intensity and frequency of the pulses depends upon the apparent density of the cultivated organism, which can be higher or lower than that of the culture medium, with consequent tendency to sedimentation or flotation.

5. The method of claim 1, wherein the tubular geometry and the presence of a culture medium buffered with a carbonate-bicarbonate buffer are exploited in order to enable complete dissolution of CO₂ and the availability of nutrients inside the entire circuit.

6. The method of claim 1, wherein the collection of the biomass produced, the maintenance of the design concentration, and the control of possible pollutant species occur through a system of differential-inclination sieves with non-clogging filtering fabric.

7. The method of claim 1, wherein said skimmers for extraction of the oxygen produced are connected to expansion vessels to prevent outflow of liquid following upon the agitation pulses.

8. A system for cultivation of photosynthetic micro-organisms through circulation of the suspension of soil micro-organism in an appropriate culture medium within tubes made of transparent plastic material, the system comprising:
a timed pumping means for pulsed movement of the culture medium inside the tube;
means of sifting comprising a plurality of differential-inclination sieves for selective separation of the biomass as well as to control the temperature of the culture medium modifying the concentration of the biomass in the culture medium so as to increase the transparency of the culture medium and reduce the solar energy absorbed;
means for input, in said culture medium, CO₂ and a buffer solution aimed at increasing absorption of CO₂ to favour growth of the organism chosen; and
skimmers distributed along the tubes traversed by the culture medium to extract the oxygen produced in the photosynthesis in a natural way.

9. The system of claim 8, wherein the photosynthetic tubes are made prevalently of plastic obtained from waste-recovery operations.

10. The system of claim 8, wherein said timed-pumping means for agitation and mixing of the culture medium is substituted by means for intermittent emptying of containers set at a higher level than the plane of lie of the system itself.

11. The system of claim 8, wherein a self-priming siphon, which enables fast emptying of containers, is set at a higher level than the plane of lie to obtain a pulsed flow.

12. The system of claim 8, comprises said plurality of differential-inclination sieves, made of non-clogging filtering fabric, for collection of the biomass produced, maintenance of the design concentration, and control of possible pollutant species.

13. The system of claim 12, wherein said sieves are designed to increase the transparency of the culture medium, consequently reducing the solar energy absorbed for controlling the temperature inside the tubes.

14. The system of claim 8, wherein said skimmers for extraction of the oxygen produced are connected to expansion vessels to prevent outflow of liquid following upon the agitation pulses.
